(19) United States
(12) Patent Application Publication
(10) Pub. No.: US 2010/0204461 A1
(43) Pub. Date: Aug. 12, 2010

(54) Bimolecular Constructs

(75) Inventors: Leslie C. BEADLING, South Plainfield, NJ (US); William H. BRAUNLIN, Highland Park, NJ (US); Roger S. CUBICCIOTTI, Montclair, NJ (US); Salvatore A.E. MARRAS, Roselle Park, NJ (US); Patricia SOTEROPOULOS, Chatham, NJ (US)

Correspondence Address: David M. McConoughey, 179 Indiana St, Maplewood, NJ, 07040-3541, US

(73) Assignees: UNIVERSITY OF MEDICINE AND DENISTRY OF NEW JERSEY, Neark, NJ (US); RATIONAL AFFINITY DEVICES, LLC, Newark, NJ (US)

(21) Appl. No.: 12/086,493

(22) PCT Filed: Dec. 13, 2006
(86) PCT No.: PCT/US2006/047523

§ 371 Date: Jun. 13, 2008

Related U.S. Application Data
(60) Provisional application No. 60/750,410, filed on Dec. 13, 2005.

(51) Int. Cl. C07H 21/04

(52) U.S. Cl. 536/24.3

(57) ABSTRACT

An immobilized bimolecular construct comprises a solid support, a first oligonucleotide and a second oligonucleotide. The first oligonucleotide is labeled at one end with a fluorophore or quencher and attached at the other end to a solid support. The second oligonucleotide is labeled at one end with a fluorophore or quencher and hybridized at the other end to the first oligonucleotide. Hybridization of the second oligonucleotide with the first oligonucleotide brings the labeled end of the second oligonucleotide in close proximity or physical contact with the labeled end of the first oligonucleotide. In one embodiment the second oligonucleotide is also attached to the solid support in proximity to the first oligonucleotide. In this embodiment, the second oligonucleotide may be first attached to the solid support and then hybridized to the first oligonucleotide or, conversely, first hybridized to the first oligonucleotide and then attached to the solid support.

### TECHNICAL FIELD

This invention relates to target-binding bimolecular constructs useful in detecting and quantifying substances in samples and subjects for applications in proteomics, diagnostics, drug discovery, medical devices, systems biology and, more generally, life sciences research and development. Compositions and methods of the invention can be used, for example and without limitation, in instrumented and noninstrumented sensors, transducers, signal processing devices and solid-phase, solution-phase, homogeneous and heterogeneous assay systems.

### BACKGROUND ART

As background, monomolecular nucleic acid-based detection constructs, such as molecular beacons, are dissimilar from the instant bimolecular constructs and disadvantageous for reasons discussed below. In the typical monomolecular beacon construct, a fluorophore and a quencher are placed on opposite ends of the same nucleic acid strand. In the nonbinding hairpin conformation, the fluorophore and quencher are in close proximity, and fluorescence emission in response to illumination is quenched. In the target-bound conformation, the fluorophore and quencher are separated by sufficient distance to circumvent quenching. In this case, illumination by light of suitable spectral qualities results in target-dependent fluorescence.

### DISCLOSURE OF VARIOUS EMBODIMENTS OF THE INVENTION

### Bimolecular Constructs of the Present Invention

An immobilized bimolecular construct of the present invention comprises a solid support, a first oligonucleotide and a second oligonucleotide. The first oligonucleotide is labeled at one end with a fluorophore or quencher and attached at the other end to a solid support. The second oligonucleotide is labeled at one end with a fluorophore or quencher and hybridized at the other end to the first oligonucleotide. Hybridization of the second oligonucleotide with the first oligonucleotide brings the labeled end of the second oligonucleotide in close proximity or physical contact with the labeled end of the first oligonucleotide. In one embodiment the second oligonucleotide is also attached to the solid support in proximity to the first oligonucleotide. In this embodiment, the second oligonucleotide may be first attached to the solid support and then hybridized to the first oligonucleotide or, conversely, first hybridized to the first oligonucleotide and then attached to the solid support.

A bimolecular construct of the present invention is described for the attachment of nucleic acid-based molecular devices to surfaces as illustrated, e.g., by immobilized molecular beacons, aptamers and tunable affinity ligands (TALs). “Bimolecular construct,” as used herein, refers to a molecular complex or its substituent components comprising at least two hybridizably linked or linkable nucleic acid-based molecules, at least one of which is capable of generating a detectable signal or attaching to a surface. The second of the at least two hybridizably linked or linkable molecules enables or facilitates surface attachment or signaling by its hybridizable partner or enhances function compared to a corresponding monomolecular construct as measured, e.g., by attachment effectiveness, efficiency, reliability, stability, sensitivity, specificity, signal-to-noise ratio, versatility, convenience, ease-of-use and/or cost effectiveness. Bimolecular constructs of the invention are advantageously used for signaling molecular interactions and/or detecting the presence or amount of a substance in a sample or subject. As such, in one embodiment of the invention, bimolecular constructs are capable of generating a signal, advantageously a signal corresponding to a specific binding event between a probe or ligand moiety of the construct and a target substance, molecule, sequence and/or cell. In another embodiment, bimolecular constructs are capable of detecting the presence and/or amount of a target substance in a sample or subject, advantageously recognizing the target with a high or controlled degree of selectivity through specific binding interactions well known in the art, e.g., nucleic acid hybridization, ligand-receptor binding, and capitalizing on the signal-generating properties of detectably labeled bimolecular constructs. Bimolecular constructs with signaling and detection functionalities have broad utility in molecular and cellular analysis; clinical, agricultural, veterinary and environmental diagnostics; military, space and forensic uses; and, more broadly, life science and industrial applications.

As used in this disclosure, “nucleic acid” and “nucleic acid-based” refers to constructs comprising a plurality of nucleotides, advantageously a sufficient number of nucleotides to participate in base-pairing, and optionally nonnucleotide monomers, polymers, spacers, linkers and the like. The term “nucleotide” includes any compound containing a heterocyclic compound bound to a phosphorylated sugar by an N-glycosyl link, any monomer capable of complementary base pairing and any analog, mimetic, congener or conjugate thereof, including modified purines and pyrimidines, minor bases, convertible nucleosides, structural analogs of purines and pyrimidines and labeled, derivatized, modified and conjugated nucleosides and nucleotides. Nonnucleotide constituents of nucleic acid-based constructs include, for example and without limitation, sequence modifiers, terminus modifiers, spacer modifiers, backbone modifications, amide linkages, achiral and neutral internucleotidic linkages and nonnucleotide bridges such as polyethylene glycol, aromatic polyamides, lipids and the like. The term “oligonucleotide” means a molecule comprising a sequence of nucleotides, typically at least three and less than about a thousand nucleotides, although the term as used herein is not intended to convey any particular limit on nucleotide sequence length. The term “nonnucleic acid” refers to a molecule or group of molecules other than a nucleic acid or oligonucleotide molecule. The term “nonoligonucleotide” refers to a molecule or group of molecules other than an oligonucleotide or nucleic acid molecule. Nonnucleic acid and nonoligonucleotide molecules are those lacking a sequence of purines, pyrimidines and/or purine or pyrimidine analogs and include, for example, peptides, proteins, sugars, carbohydrates, lipids, inorganic molecules, purine and pyrimidine monomers and naturally occurring and synthetic monomers, dimers, trimers, oligomers, polymers and analogs, mimetics, conjugates and complexes thereof. The term “proximity” with regard to fluorophore/quencher interaction refers to a distance sufficiently small (the “energy transfer distance”) to allow detectable fluorophore-quencher energy transfer, advantageously a distance in the range of the Forster energy transfer distance (“Forster distance”) or a small multiple thereof that allows for energy transfer efficiency of at least about 10%. The Forster distance is based on the principle of fluorescence resonance energy transfer or FRET. Fluorescence resonance energy transfer (FRET) is the distance-dependent transfer of excited state energy from a donor fluorophore to an acceptor fluorophore. The Förster distance is a characteristic distance for energy transfer and provides a spectroscopic ruler. The Förster distance is defined as the distance at which FRET is 50% efficient.

For the purposes of this application, molecular beacons are defined as hairpin-forming nucleic acid-based ligands that, upon binding to target, switch from a quenched conformation to one that fluoresces. Typically, the targets of molecular beacons as described in the art are nucleic acid sequences complementary to the loop region of the molecular beacon hairpin. The hairpin loop is designed to contain a probe sequence optimal for specific hybridization to the target of interest. Bimolecular constructs of the present invention comprehend and incorporate molecular beacon-like hairpin probe regions for specific detection of nucleic acid targets as well as other nucleic acid-based ligands that recognize and detect a diverse assortment of nucleotide and nonnucleotide molecules through hybridization and nonhybridization-based interactions with target molecules. The term “beacon,” as used herein, is occasionally used to refer to a beacon-like structure, component, region or functional element of fluorophore- and quencher-labeled hairpin probes known in the art as molecular beacons. For convenience, terms like “beacon” and “beacon moiety,” e.g., are sometimes used in reference to a hairpin oligonucleotide or fluorophore- or quencher-labeled hairpin oligonucleotide that lacks the full complement of features required for target-dependent signal generation. For example, “beacon,” and “beacon moiety” may be used as generic terms in reference to, e.g., a hairpin-forming oligonucleotide comprising a bimolecular construct or a hairpin-containing precursor of a bimolecular construct.

Bimolecular constructs can be designed to detect and quantify substances over a wide range of sizes, shapes and compositions, including, e.g., cells, cell surface markers, subcellular structures, liposomes, vesicles, microorganisms, nanoparticles, macromolecules, multimers, natural and synthetic polymers, oligomers, monomers and small molecules. Targets may include, for example and without limitation, nucleic acids, proteins, peptides, antibodies, antigens, haptens, carbohydrates, drugs, pharmacophores (including biological, bioderived, bioinspired and synthetic drug candidates, leads, prospects, analogs, congeners, mimetics, agonists, antagonists, competitors and the like), hormones, growth factors, autocoids, transmitters, vitamins, metabolites, cofactors, food pathogens, toxins, environmental pollutants, industrial contaminants, infectious agents, biomolecular complexes (e.g. ribonucleoprotein complexes, multimeric proteins and protein complexes, lipid and lipoprotein particles and protein-carbohydrate complexes), cell surfaces, viruses, and other complex biological targets. Small molecules, as distinct from macromolecules, are intended to comprehend molecules having a number-average/weight-average molecular weight of under about 5,000 Daltons and more typically under about 2,000 Daltons, though the term can also be applied to low molecular weight polymers such as oligonucleotides, oligopeptides, oligosaccharides and the like, for which it is difficult to justify a specific molecular weight cutoff between, say, 5,000 Daltons and 10,000 Daltons. For purposes of this disclosure, “small molecules” shall mean those having a molecular weight less than about 5,000 Daltons with discretion as needed in the case of selected oligomeric species. Biomolecular complexes are intended to comprehend noncovalent associations of biologically occurring molecules, including proteins, nucleic acids, carbohydrates, small molecules and associated ions. Examples include ribosomes and other ribonucleoprotein complexes, biologically functional protein complexes in muscles, the cytoskeleton, secretory processes and nonfunctional biomolecular aggregates (e.g., prion protein precipitates and Alzheimer plaques.) Other complex biological targets include the extracellular biological matrix, biofilms, and other complex associations of living cells, colonies of cells, and associated biopolymer matrices. Proteins are intended to comprehend glycoproteins and lipoproteins.

Molecular beacon target binding sequences can be naturally occurring, rationally designed, or discovered by a combinatorial process such as SELEX. When used in reference to an immobilized detection reagent or solid phase binding assay, the term “surface” refers to a support, advantageously a solid, semi-solid or insoluble substance, material, or matrix, to which molecules can be attached, e.g., for the purpose of distinguishing surface-bound molecules and complexes from solution-phase molecules and complexes. The term “support” refers to the surface/structure to which molecules can be attached or otherwise immobilized, associated, localized and/or insolubilized.

As stated above, in the typical monomolecular construct, a fluorophore and a quencher are placed on opposite ends of the same nucleic acid strand. In the “unbound” (target-free) hairpin conformation that prevails in the absence of target, fluorophore and quencher are in close proximity, and excitation-induced fluorescence is quenched. In the target-bound conformation, the fluorophore and quencher are spatially separated by the intervening probe-target complex, and fluorescence occurs upon illumination by light of suitable wavelength.

In the bimolecular construct, the fluorophore and the quencher are placed on separate strands, thereby providing key advantages over attachment methods using monomolecular beacons. Because use of the preferred bimolecular construct results in the projection of a duplex structure from the attachment surface, a more rigid spacer separates the fluorophore and the quencher from the modified surface. Interaction between the fluorophore and the quencher is therefore favored over interaction between the fluorophore or the quencher and the surface. As a consequence of the bimolecular design, which limits the interaction of fluorophore and/or quencher with the surface, reduced background fluorescence is obtained for bimolecular compared to unimolecular beacons. Another advantage of the bimolecular construct is that surface attachment can occur through both of the duplex stands. The bimolecular construct can thus be attached to the surface by (at least) two covalent bonds, rather than just one. A major advantage of attaching both strands is that rigorous washing procedures can be performed following immobilization to remove nonspecifically bound fluorescent moieties from the surface, without risking removal of the hairpin-forming oligonucleotide from the bimolecular construct. Finally, in the case where the target is a protein or other nonnucleic acid molecule, the bimolecular construct allows greater control over the designed placement and target-dependent separation of the fluor and quencher moieties.

### Introduction—Molecular Beacons, Aptamers and Tunable Affinity Ligands

1. Principle of Operation of Molecular Beacons

Molecular beacons are nucleic acid probes that undergo a conformational change and fluoresce brightly when they bind to their target (See, for example, Tyagi and Kramer, 1996; Tyagi, Bratu et al., 1998). These probes are single-stranded nucleic acids that form a stem-and-loop structure (FIG. 1). In the most common configuration, as a hybridization probe, the loop portion of the molecule is complementary to a target nucleic acid sequence, and is located between two arm sequences that are complementary to each other. The arms bind to each other to form a double-helical stem hybrid forming a hairpin structure. A fluorophore is covalently linked to one end of the oligonucleotide and a nonfluorescent quencher moiety is covalently linked to the other end of the oligonucleotide (See, for example, Tyagi, Bratu et al., 1998; Marras, Kramer et al., 2002). The stem hybrid brings the fluorophore and quencher in close proximity, allowing energy from the fluorophore to be transferred directly to the quencher through static quenching (Marras, 2005). When a molecular beacon encounters a target molecule, it spontaneously reorganizes, forming a probe-target hybrid that is longer and more stable than the stem hybrid, forcing the stem hybrid to dissociate. The fluorophore and the quencher thus move away from each other, and the beacon becomes fluorescent. In practice, the length of the probe sequence is chosen so that it will form a stable hybrid with its target sequence at assay temperatures, whereas the arm sequences are chosen so that they will form a stable stem hybrid when there is no target present. See, FIG. 1, showing that when the probe sequence in the loop of a molecular beacon binds to a target sequence a conformational reorganization occurs that restores the fluorescence of a quenched fluorophore. (See also, for example, Marras, 2003a).

Since molecular beacons are dark (nonfluorescent) when not hybridized and brightly fluorescent when hybridized to their targets, the course of hybridization can be followed in real time with a spectrofluorimeter. FIG. 2 shows the results of an experiment in which the addition of an excess of complementary oligonucleotide target to a solution of molecular beacons caused a 100-fold increase in fluorescence intensity. See FIG. 2, illustrating functional characterization of a molecular beacon by adding a complementary oligonucleotide target. (See also, for example, Marras, Kramer et al., 2003b).

Just as in any other nucleic acid hybridization reaction, the binding of a molecular beacon to its target follows second order kinetics, and the rate of the reaction depends on the concentration of the probe, the concentration of the target, the temperature, and the salt concentration. Under in vitro and in vivo assay conditions, in which the molecular beacon concentration is chosen so that they will always be more abundant than the target, hybridization is spontaneous and rapid, reaching completion in only a few seconds, and the intensity of the resulting fluorescence is linearly proportional to the amount of target present.

Since the introduction of molecular beacons, they have been used in a number of studies that would have been far more difficult to perform with conventional hybridization probes. Molecular beacons are able to monitor the progress of any amplification reaction where either single-stranded or double-stranded nucleic acids are formed. Real-time monitoring of the synthesis of DNA or RNA sequences have been developed for PCR, NASBA, rolling circle amplification and the isothermal ramification amplification method (See, for example, Marras, 2003b). In addition, molecular beacons have been used to detect the movement of specific RNAs in living cells (See, for example, Bratu, Cha et al., 2003). Other studies use molecular beacons to measure enzymatic activities, duplex and triplex formation in nucleic acids, and interactions between proteins and nucleic acids (See, for example, Marras, Kramer et al., 2003a)).

2. Molecular Beacons with Nonnucleic Acid Targets.

Aptamers are nucleic acid ligands that have been discovered by the combinatorial process known as SELEX (See, for example, Brody and Gold, 2000; Famulok and Mayer, 1999; Wilson and Szostak, 1999). Aptamer beacons are molecular beacons that are constructed using known aptamers and are designed to fluoresce in the presence of target (e.g. a protein) and to be quenched in the absence of target. Ellington and coworkers have designed monomolecular aptamer beacons based on the well-studied thrombin aptamer that fluoresce in protein-binding G-quadruplex form and that are quenched when in the competing hairpin form (See, for example, Hamaguchi, Ellington et al., 2001). Beacons can also be derived using naturally occurring protein-binding nucleic acid sequences, for example in gene-regulatory regions of the chromosome. We will discuss below particular examples of both naturally occurring sequences and aptamer sequences that can be integrated into protein-binding molecular beacon design.

3. Tunable Affinity Ligands (TALs).

TALs are ligands defined by the following properties:
a) They can take on two or more conformations that differ in target binding affinities. In the simplest case, TALs exist in two distinct conformations. One conformation binds target tightly and specifically, and the other conformation manifests weaker, nonspecific binding to target.
b) Partitioning among accessible conformations can be controlled by modest changes in solution conditions. The environmental effectors of switching between TAL active and inactive conformations include K⁺, for quadruplex forming TALs, Mg²⁺ for triplex and junction forming TALs, and pH for TALs that involve the i-motif, triple-helix formation, or other structures involving cytosine protonation.
c) Since the ligand binding affinity depends strongly on conformation, modest changes in solution conditions result in large changes in ligand binding affinity. For example, a number of proteins are known to bind specifically to quadruplex nucleic acid structures (See, for example, Cogoi, Quadrifoglio et al., 2004; Dapic, Abdomerovic et al., 2003; Jing, Li et al., 2003; Lin, Shih et al., 2001; Rangan, Fedoroff et al., 2001; Siddiqui-Jain, Grand et al., 2002). By varying the ratio of K⁺ to Li⁺ in solution, we can modulate the quadruplex-hairpin equilibrium of our TALs, and thereby the affinity of these TALs for target proteins.
d) Balancing the conformational equilibria of TALs results in an enhancement of selectivity of target binding. A thermodynamic analysis of this effect has been articulated for molecular beacons, but is equally applicable for TALs (See, for example, Bonnet, Tyagi et al., 1999).
e) The binding conformation of TALs can be biologically derived, e.g. as a duplex binding site of gene-regulatory proteins, or as a quadruplex forming region of biological significance. The binding region can also be an aptamer arrived at by SELEX methodology. Finally, the binding conformation can be derived by any combination of procedures involving rational design followed by screening, followed by optimization.

4. Quadruplex-Hairpin Tunable Affinity Ligands (TALs).

As a specific example of TALs, we have focused on nucleic-acid based ligands that can partition between quadruplex and hairpin forms. The partitioning between quadruplex and hairpin depends strongly on the presence of ions such as K⁺, which coordinate specifically with, and thereby stabilize, quadruplex structures. Bulky ions such as Li⁺ are unable to coordinate specifically, and will therefore shift the equilibrium toward the hairpin. The binding conformation of a given Tunable Affinity Ligand (TAL) can be biologically derived, e.g. from quadruplex-forming sequences such as genomic G-rich regions, including telomeres, the c-MYC promoter region, and fragile X expansion regions. The binding conformation can also be derived from aptamers, arrived at by the SELEX methodology, e.g. the thrombin aptamer, or the aptamer for the receptor activator of NF-κB (RANK). Finally, the binding conformation can be derived by any combination of procedures involving rational design followed by screening, followed by optimization.

5. Tunable Affinity Ligand (TAL) Beacons.

Tunable Affinity Ligand (TAL) beacons are TALs that exist in either a quenched conformation or an unquenched conformation. We define standard TAL beacons as molecules for which the unquenched conformation shows specific target binding affinity, while the quenched conformation binds the same target with reduced affinity. Molecules for which the quenched conformation binds target specifically and the unquenched conformation binds target with reduced affinity we define as reverse TAL beacons. One example of a TAL beacon design is a monomolecular construct where quencher and fluorophore are on opposite ends of the same molecule, and where one set of conditions favors a stem-loop hairpin conformation, and contact-quenching of fluorescence (See, for example, Hamaguchi, Ellington et al., 2001). Under other conditions, the TAL shifts to a quadruplex conformation that favors target binding, with a separation of fluorophore and quencher:

6. Enhanced Specificity of Molecular Beacons.

Hybridization-based molecular beacons recognize their target nucleic acids with greater specificity than linear oligonucleotide probes (See, for example, Tyagi, Bratu et al., 1998; Marras, Kramer et al., 1999; Bonnet, Tyagi et al., 1999). In a similar manner, protein-binding molecular beacons recognize their target proteins with greater specificity than nonswitchable aptamers, as a consequence of balancing the conformational equilibrium of an active form with a hairpin structure (See, for example, Bonnet, Tyagi et al., 1999). When a molecular beacon binds to its target sequence, the probe-target hybrid occurs at the expense of the hairpin. When a protein-binding molecular beacon binds to its target protein, the equilibrium shifts from an inactive hairpin conformation to an active conformation.

Molecular beacons are designed so that over a wide range of temperatures, only perfectly complementary probe-target hybrids are sufficiently stable to open the stem structure. Mismatched probe-target hybrids do not form except at substantially lower temperatures (See, for example, Marras, Kramer et al., 1999; Bonnet, Tyagi et al., 1999). Therefore a relatively wide range of temperatures exist in which perfectly complementary probe-target hybrids elicit a fluorescent signal while mismatched molecular beacons remain dark. Consequently, assays using molecular beacons robustly discriminate targets that differ from one another by as little as a single nucleotide substitution. This high specificity allows detection of a small proportion of mutant DNA in the presence of an abundant wild-type DNA (See, for example, Szuhai, Ouweland et al., 2001).

Similarly, protein-binding molecular beacons can be optimized so that only specific target complexes are favored, and related protein targets will only form at lower temperatures. This enhanced specificity can be used to discriminate protein binding partners even if the inherent free energy of binding is very similar. In summary, an analog can be made between the balancing of hairpin vs. linear duplex equilibria in nucleic acid target detection, and the balancing of hairpin vs. protein binding equilibria in protein target discrimination with molecular beacons. In the former case, hairpin probes allow enhanced discrimination between fully complementary targets vs. targets with a single mismatch. In the latter case, hairpin probes allow enhanced discrimination among proteins with similar, but not identical binding sites. In both cases, the enhanced discrimination comes at the cost of decreased overall binding.

### Introduction—Beacons on Surfaces.

In solution, conventional molecular beacons show exquisite sensitivity for single base-pair mismatches, and do not require the labeling of target. Fluorescence enhancements are generally around 25×, and enhancements of up to 200× have been reported (See, for example, Yao and Tan, 2004). Over the past few years, several groups have tested molecular beacon arrays for multiplexed SNP detection (See, for example, Yao and Tan, 2004; Culha, Stokes et al., 2004; Steemers, Ferguson et al., 2000; Wang, Li et al., 2002). These studies have demonstrated varying degrees of success. As outlined by Beaucage, the requirements for the successful application of arrayed oligonucleotides include the following: 1) chemically stable attachment chemistry, 2) a sufficiently long linker to minimize steric interferences, 3) hydrophilic linker to ensure solubility in aqueous solution, and 4) minimal nonspecific binding to the glass surface (See, for example, Beaucage, 2001). The requirements for molecular beacon arrays are even more stringent. First, nonspecific interactions of hydrophobic dyes with both surfaces and linkers need to be minimized. Such interactions could result in a partial destabilization of the quenched hairpin state, which could in turn give a high background fluorescence. An additional concern for surface-attached molecular beacons would be maintaining the high discrimination ratio for single nucleotide mismatches that is obtained in solution.

In fact, and in contrast to the solution situation, immobilized molecular beacons in array studies do tend to suffer from a high fluorescence background, with fluorescence enhancements in the single digit range. As we have determined that titrating linkers into solution-phase molecular beacon assays has little effect on assay performance, the high fluorescent background noted in array-based assays appears to result from surface interactions rather than linker interference.

A variety of immobilization methods and surface modifications have been used for the attachment of oligonucleotides in general and molecular beacons in particular to glass slides (See, for example, Beaucage, 2001). These methods include a) robotic deposition of oligonucleotides on polylysine or aminosilane-coated surfaces, b) covalent attachment of oligonucleotides through aminoalkane linkers to aldehyde or epoxide modified glass surfaces, c) physical adsorption of avidin on glass-slides followed by noncovalent attachment of DNA via a biotin linker, d) reductive coupling of amino-linked oligonucleotides to polyacrylamide or agarose gels, e) attachment of oligonucleotides to gold surfaces either directly using thiol-linkers or indirectly to self-assembled monolayers (SAMS) on gold surfaces using biotin-streptavidin cross-links, f) attachment to a polyelectrolyte multilayer surface via biotin-streptavidin linkage (See, for example, Kartalov, Unger et al., 2003).
a) Polylysine or aminosilane-coated surfaces. Microarrays of cDNAs are often generated by robotic deposition of PCR-amplified DNAs coated with poly-L-lysine or with aminosilanes. This approach relies on the nonspecific electrostatic interaction of negatively charged DNA phosphates with positively charged groups on the slide surface. Such interactions reduce the conformational freedom of the bound DNA, and thus limit the accessibility of complementary probe sequences for target. If applied to the spotting of molecular beacons, such interactions can potentially trap molecular beacons in unquenched conformations, and reduce the discrimination ratio for single-nucleotide mismatches. Although direct spotting of molecular beacons on positively charged surfaces may be the simplest method, it is unlikely to provide either a high signal to noise ratio or a good discrimination ratio. The primary utility of molecular beacon studies on such surfaces is to provide a negative baseline for molecular beacon performance. The corresponding positive baseline is molecular beacon behavior in solution.
b) Covalent attachment through aminoalkane linkers. Aldehyde-derivatized glass slides prepared from silanization are easily prepared, and commercially available. Using commercially available phosphoramidites, molecular beacons may be synthesized with aminohexyl linkers projecting from the 5′ or 3′ ends, or projecting off of thymines within the DNA sequence. When aminohexyl modified oligonucleotides are spotted onto aldehyde-derivatized slides, they become covalently attached via Schiff's base formation. Subsequent reduction with NaBH₄ leads to a stable covalent linkage and conversion of remaining aldehydes into hydroxyls. Alternatively, a stable hydrophilic surface can be produced through a milder reaction with NaCNBH₃ plus ethanolamine to cap the remaining surface aldehydes. The coating of hydroxyl groups remaining on the chip surface following either procedure acts to reduce nonspecific hydrophobic associations of DNA bases or of bulky hydrophobic dyes. Highly reactive, epoxide-coated slides can be similarly derivatized, and capped to minimize hydrophobic interactions.
c) Physical adsorption of avidin and noncovalent attachment of biotinylated DNA. Avidin binds tightly to glass by physical adsorption and this interaction can be further stabilized by treatment with glutaraldehyde. Once bound, accessible biotin binding sites allow the tight attachment of biotinylated DNA, which can be synthesized using commercially available phosphoramidites. Though this approach has been used with some modest degree of success, it is problematic in that avidin is a fairly basic protein, and oligonucleotides anchored to avidin are likely to interact nonspecifically with this protein, potentially resulting in both an increased fluorescent background and a decrease in single molecule discrimination. The pH dependent fluorescence background observed for slides prepared by physical adsorption of avidin quite likely reflects such nonspecific association of avidin with attached molecular beacons (See, for example, Yao and Tan, 2004).
d) Molecular beacons covalently linked to hydrogels. Both polyacrylamide and agarose gel coatings have been applied to glass slides and covalently derivatized with oligonucleotides. Initially, the preparation of such coatings represented a moderate technical challenge, and was confined to a few labs (See, for example, Beaucage, 2001; Timofeev, Kochetkova et al., 1996; Khrapko, Lysov Yu al., 1989; Khrapko, Lysov Yu et al., 1991). Recently, a considerably simpler method of preparation of derivatized agarose gels has been proposed (See, for example, Wang, Li et al., 2002; Afanassiev, Hanemann et al., 2000). The advantages of hydrogels are 1) high binding capacity due to the three-dimensional nature of the gel, and 2) a more solution-like hybridization environment. A direct comparison of molecular beacons covalently linked through 6-amino groups to aldehyde slides and linked to agarose gel film suggested that the latter method of immobilization was indeed superior in terms of decreased fluorescence background and enhanced specificity for single base-pair discrimination (See, for example, Wang, Li et al., 2002).
e) Attachment of oligonucleotides to gold surfaces. Gold surfaces are often used for biopolymer attachment in the context of surface plasmon resonance, electrochemical, or other nonfluorescent detection methods. DNA oligonucleotides modified with a C6 thiol group may be immobilized through self-assembly onto gold surfaces. On bare gold, thiol-modified single-stranded DNA molecules shorter than about 24 nucleotides organize in extended conformations, whereas longer molecules form more of a blob-like layer. Since amines are known to absorb weakly to gold, this result suggests multiple weak contacts between DNA amines and the surface of the gold. Treatment of thiol-DNA surfaces with 6-mercapto-1-hexanol (MCH) displaces these weak absorptive interactions, allowing the longer DNA sequences to extend more fully into solution, and be more accessible to target (See, for example, Steel, Levicky et al., 2000). Gold surfaces have several key advantages in the context of molecular beacon studies (See, for example, Steel, Levicky et al., 2000; Du, Disney et al., 2003). First, the self-assembled monolayer (SAM) of MCH provides a hydrophilic surface that may be used to reduce the strength or degree of attraction of hydrophobic dye conjugates. Second, the gold surface itself may act as a quenching agent for fluorescent dyes, and thus eliminate the requirement for doubly labeling the molecular beacon hairpin (See, for example, Du, Disney et al., 2003). Third, the DNA molecules in the SAM will tend to repel each other electrostatically, and will thus naturally be spread out on the surface of the monolayer. Also, the optimum ratio of DNA to MCH can be determined by the input mixing ratios, thereby providing an additional level of quality control. Finally, the SAM on gold provides significant flexibility for compositional control and attachment chemistries. For example, 6-mercapto-1-hexanoic acid can be introduced to modulate the final surface charge of the SAM in order to repel negatively charged oligonucleotides. Biotin terminated thioalkanes can be used to trap streptavidin, which in turn can be used to bind biotinylated oligonucleotides.

Hybrid surfaces comprising hydrogels layered on top of gold surfaces provide an additional level of control over surface properties. The standard surface for surface plasmon resonance (SPR) studies is a gold surface that is derivatized with a matrix of carboxymethylated dextran (See, for example, Lofas and Johhsson, 1990). This surface has shown excellent compatibility with a variety of biopolymers, including oligonucleotides, and represents an attractive surface for bimolecular construct immobilization.
f) Attachment to a polyelectrolyte multilayer surface. Sequential layering of polycations and polyanions on surfaces allows the formation of thin films of polyelectrolyte multilayers (See, for example, Decher, 1997). Such surfaces have many useful features. For example, by varying the charge on the final layer, repulsive electrostatic interactions can be engineered to provide very low specific adsorption characteristics for charged biomolecules. Kartalov et al used multilayers of polyethylene amine/polyallylamine and polyacrylic acid to anchor DNA through biotin-streptavidin bonding (See, for example, Kartalov, Unger et al., 2003). The final layer in their film was polyacrylic acid, which provided a DNA-repellant, (a negatively charged surface) that functioned to suppress nonspecific binding to facilitate single-molecule fluorescence studies (See, for example, Kartalov, Unger et al., 2003).

### EXEMPLARY EMBODIMENTS OF THE PRESENT INVENTION

1. Design Features of Bimolecular Detection Constructs for Hybridization Analysis.

One design the bimolecular construct of the present invention is illustrated in FIG. 3. An anchor strand allows linkage of the beacon moiety to the surface via a 5′ linker, and positions the quencher on the 3′ end. A probe strand hybridizes to the anchor via its 5′ end, and may also have a linker group on its 5′ end to facilitate surface attachment. The 5′ linker on the anchor strand can be, e.g., a hexylamine sequence, that allows covalent attachment by Schiff's base formation with aldehyde groups on the surface. The beacons strand has a fluorophore at the 5′ end and may have an additional linker at the 3′ end for attachment to the slide surface. The beacon strand is designed to form a stem-loop structure in the absence of target, and to open up, separating the fluorophore and quencher in the presence of target. See FIG. 3, which illustrates a novel molecular beacon with 5′ fluorophore and 3′ linker for attachment to slide surface and complementary quencher bearing linker.

2. Design Features of Bimolecular TAL Beacons for Protein Targeting.

In the present invention we have introduced an analogous TAL beacon design (FIG. 4) where the quencher is attached to a separate stem structure that anchors the fluorophore-containing TAL beacon to a surface. See FIG. 4 for a novel TAL beacon with 5′ fluorophore and 3′ linker for attachment to slide surface and complementary quencher bearing linker. At A in FIG. 4 the anchor sequence, with 3′ quencher is attached via a 5′ amino functionality to an amine-reactive surface. At B in FIG. 4 the aptamer functionality is hybridized to the anchor under conditions favoring hairpin formation (e.g. LiCl solution). At C in FIG. 4 the TAL is switched to a protein-binding conformation (here, a quadruplex) under other conditions (e.g. KCl solution). At D in FIG. 4 protein binding to the active TAL conformation shifts the equilibrium toward that conformation.

3. Advantages of Bimolecular Constructs Compared to Monomolecular Beacons.

By placing the fluorophore and the quencher on separate strands, with complementary bases holding them together, a key advantage is obtained over attachment methods using monomolecular beacons: Since a duplex structure projects from the surface, a more rigid spacer separates the fluorophore and the quencher from the modified surface. As a consequence, interaction between the fluorophore and the quencher is favored compared to interaction between the fluorophore or quencher and the surface. Because of the bimolecular design, which limits the interaction of fluorophore and/or quencher with the surface, reduced background fluorescence can provide enhanced signal-to-noise ratios compared to unimolecular beacons. Another advantage is that the ratio of anchor strand and beacon strand can be optimized in order to maximize signal compared to background. A final advantage is that it is simpler, more efficient, and more economical to synthesize the quencher and fluorophore on opposite strands. For a monomolecular beacon, it is necessary to synthesize molecules that have a) a linker group for surface attachment, b) an internal quencher or fluorophore and c) a terminal quencher or fluorophore. For bimolecular constructs, each oligonucleotide need only have one terminal linker for surface attachment, and one terminal fluorophore or quencher.

### EXAMPLES

### Example 1

### Bimolecular Probes for Nucleic Acid Detection in Solution

A fluorescein labeled hairpin DNA Oligonucleotide, HP2, with a ten base-pair linker sequence was machine synthesized and HPLC purified. The sequence of HP2 was:

5′ FAM - CGTCG ACC ATG ATC GGC GGC CGACG CTGTG
CTCGC - 3′

The underlined stretches in this sequence represent arm sequences that form the stem structure of the hairpin in the absence of complementary nucleic acid target. An anchor-oligo sequence representing the linear complement to the ten base-pair linker sequence of HP2 was also synthesized and HPLC purified. The sequence of this anchor-oligo was:

5′ - GCG AGC ACA G - BHQ2 - 3′

Finally, the target oligonucleotide complementary to the loop region of HP2 was synthesized and purified. The target oligo sequence was:

5′ - GCC GCC GAT CAT GGT - 3′

The fluorescence background of 150 μl of a 1 mM MgCl₂, 20 mM Tris-HCl, pH 8.0 solution was determined, using 491 nm as the excitation wavelength and 515 as the emission wavelength. 10 μl of 1 μM HP2 was added to this solution and the new level of fluorescence was recorded. A two-fold molar excess of anchor oligo was added and the decrease in fluorescence was monitored until it reached a stable level. Finally, a five-fold molar excess of target oligo was added and the increase in fluorescence was monitored.
As shown in FIG. 5, these experiments demonstrate that our bimolecular construct behaves as a molecular beacon for the solution monitoring of hybridization. See, FIG. 5, showing solution characterization of a bimolecular probe with 5′ fluorophore and 3′ linker for attachment to slide surface and complementary quencher.

### Example 2

### Bimolecular 2′O-Methyl Probe for Detection of Complementary microRNA

A Dabcyl labeled 2′O-methyl hairpin oligonucleotide, HP3, with a ten base-pair linker sequence was machine synthesized and HPLC purified. The sequence of HP3 was:

5′ CUG CUA CGU G -CUCG AC CAC ACA ACC
CGAG -DABCYL 3′

The underlined stretches in this sequence represent arm sequences that form the stem structure of the hairpin in the absence of complementary nucleic acid target. A 2′O-methyl anchor-oligo sequence representing the linear complement to the ten base-pair linker sequence of HP3 was also synthesized and HPLC purified. The sequence of this anchor-oligo was:

5′ - FAM-CAC GUA GCA G - 3′

Finally, a target RNA sequence corresponding to the let7b miRNA was synthesized. The let7b sequence was fully complementary to the loop sequence in HP3.
The interaction of FAM-labeled anchor oligo with the Dabcyl-labeled let7b probe gives a decrease in fluorescence as hairpin formation brings the FAM and Dabcyl groups into near proximity. As let7b target is added, quenching is reduced and fluorescence increases as binding of the let7b target opens the hairpin and separates the FAM and Dabcyl groups. See, FIG. 6, providing solution characterization at room temperature of a bimolecular construct comprising a 5′ FAM labeled 2′O-methyl anchor RNA and a 3′ dabcyl labeled 2′ O-methyl RNA probe complementary in the hairpin loop region to let7b RNA.

### Example 3

### Bimolecular 2′O-Methyl Probe for Single Base Pair Discrimination of MicroRNA

A Dabcyl labeled 2′O-methyl hairpin oligonucleotide, HP3, with a ten base-pair linker sequence was machine synthesized and HPLC purified. The sequence of HP3 was:

5′ CUG CUA CGU G -CUCG AC CAC ACA ACC
CGAG -DABCYL 3′

The underlined stretches in this sequence represent arm sequences that form the stem structure of the hairpin in the absence of complementary nucleic acid target. A 2′O-methyl anchor-oligo sequence representing the linear complement to the ten base-pair linker sequence of HP3 was also synthesized and HPLC purified. The sequence of this anchor-oligo was:

5′ - FAM-CAC GUA GCA G - 3′

Finally, RNA sequences corresponding to the miRNAs let7a, let7b, let7c and let7f were synthesized. The let7b sequence was fully complementary to the loop sequence in HP3. The target oligo sequences were:

Let7a:
5′ U GAG GUA GUA GGU UGU AUA GUU 3′
Let7b:
5′ U GAG GUA GUA GGU UGU GUG GUU 3′
Let7c:
5′ U GAG GUA GUA GGU UGU AUG GUU 3′
Let7f:
5′ U GAG GUA GUA GAU UGU AUA GUU 3′

The mismatches with respect to the probe let7b sequence are underlined. The bimolecular let7b construct of FAM-labeled anchor oligo and Dabcyl-labeled let7b probe easily discriminates between targets that differ by a single base pair. Notably, under the conditions of these experiments, targets with more than one mismatch have no measurable effect on the fluorescence of the bimolecular construct. See, FIG. 7, illustrating solution characterization of the temperature dependence of a bimolecular construct comprising 800 nM 5′ FAM labeled 2′O-methyl anchor RNA and 2 μM 3′ dabcyl labeled 2′ O-methyl let 7B RNA probe in the presence of let 7A (two mismatches), let 7B (fully complementary), let 7C (single mismatch) and let 7F (three mismatches) target molecules at concentrations of 8 μM each.

### Example 4

### Bimolecular TAL Beacons for Protein Analysis

### Titration with Complementary DNA

Recent data that we have obtained for TAL beacon constructs demonstrate the utility of TALs for protein profiling applications. The thrombin aptamer beacon constructs that we have examined are designed according to the features shown in FIG. 4. See, FIG. 4, illustrating a novel TAL beacon with 5′ fluorophore and 3′ linker for surface attachment and complementary anchor with 3′ quencher and 5′ linker. In one construct, the anchor sequence was 5′NH₂-(CH₂)₆-CACGTAGCAG-Dabcyl³′ and the hairpin-forming TAL construct (TAL2) was 5′Cy³-GGTTGGTTTGGTTGGCAACCTCTGCTACGTG³′. TAL2 was designed to base pair with the anchor sequence under the appropriate ionic conditions. In the hairpin form, the molecule should be quenched, whereas in the quadruplex form, it should fluoresce. In solution, we found that a 277 nM solution of TAL2 had a measured fluorescence intensity of about 5.4×10⁵ cps. In the presence of a 1.5 fold excess of anchor, the measured fluorescence intensity decreased 30-fold, to about 2×10⁴ cps. See, FIG. 8, showing the effect of a 1.5 fold excess of complement on the fluorescence intensity of the TAL beacon. Subsequent addition of the complementary sequence 5′d(CCAACCAAACCAACC) resulted in a dramatic increase in fluorescence, to a maximum value or about 1.6×10⁵ cps. This fluorescence behavior was observed under a variety of solution conditions, and was independent of the presence or absence of K⁺ in solution. To illustrate this point, we compared measurements carried out in 100 mM KCl with measurements in 100 mM LiCl. See, FIG. 8, demonstrating the effect of a 1.5 fold excess of complement on the fluorescence intensity of the TAL beacon. The results of these measurements demonstrated that, even in presence of 100 mM KCl, the thermodynamically stable structure for our construct is the hairpin form. Quite likely, the additional stabilization of the physical interaction between Cy3 and Dabcyl acted to shift the equilibrium away from the quadruplex, and toward the hairpin form (Marras, Kramer et al., 2002). Note however that even though the favored form was the hairpin in both LiCl and KCl, the kinetics of the association of complement was significantly slower in the presence of KCl than was observed in the presence of LiCl. This result suggests that the association of complement and TAL construct likely goes through a quadruplex intermediate in KCl solution, but not in LiCl solution.

### Example 5

### Bimolecular TAL Beacons for Protein Analysis

### Recognition of α-Thrombin

α-thrombin was obtained from Haematologic Technologies, Inc. and used without further purification. Oligonucleotides were machine synthesized and HPLC purified. A solution containing 277 nM TAL2 and 1.5 fold molar excess of Dabcyl anchor was prepared in buffer containing 10 mM KCl, 5 mM MgCl₂, and 12.5 mM Tris Acetate, pH 6.5, and titrated with a 10-fold excess of α-thrombin. Even though the quenched hairpin dominated in the absence of complementary DNA or protein target, titration with α-thrombin induced a dramatic increase in fluorescence, suggesting protein-induced stabilization of the quadruplex form. See, FIG. 9, which shows the results of adding a 10 fold excess of α-thrombin on the fluorescence intensity of the TAL2 beacon construct. The kinetics of this increase were very slow, with a t_{1/2} under these conditions of about 30 minutes.

### Example 6

### Bimolecular Tunable Affinity Ligand (TAL) Beacons for Protein Analysis Protein Concentration Dependence

α-thrombin was obtained from Haematologic Technologies, Inc. and used without further purification. Oligonucleotides were machine synthesized and HPLC purified. A solution containing 277 nM TAL2 and 1.5 fold molar excess of Dabcyl anchor was prepared in buffer containing 10 mM KCl, 5 mM MgCl₂, and 12.5 mM Tris Acetate, pH 6.5, and titrated with increasing concentrations of α-thrombin. When the TAL2 beacon was titrated with α-thrombin, both the limiting fluorescence and the kinetics of fluorescence increased strongly with increasing total concentration of protein. See, FIG. 10, showing α-thrombin concentration dependence of the fluorescence from the TAL2 beacon. The TAL concentration was 277 nM. α-thrombin was titrated to ratios of added α-thrombin to TAL of 1:1, 10:1 and 100:1.

### Example 7

### Bimolecular Tunable Affinity Ligand (TAL) Beacons for Protein Analysis Discrimination Among Closely Related Thrombin Variants

α-thrombin, β-thrombin and γ-thrombin were obtained from Haematologic Technologies, Inc. and used without further purification. Oligonucleotides were machine synthesized and HPLC purified. A solution containing 277 nM TAL2 and 1.5 fold molar excess of Dabcyl anchor was prepared in buffer containing 10 mM KCl, 5 mM MgCl₂, and 12.5 mM Tris Acetate, pH 6.5, and titrated with each of the thrombin variants. When a constant concentration of TAL2 beacon was titrated to a constant ratio of 100:1 protein to beacon, clear differences were apparent among the closely related variants, α-thrombin, β-thrombin and γ-thrombin. See, FIG. 11, which compares the effects of 100:1 molar ratios of α-, β- and γ-thrombin on the dilution-corrected fluorescence of the TAL beacon. The total concentration of TAL1 was 277 nM. The solution contained 10 mM KCl, 5 mM MgCl₂, and 12.5 mM Tris Acetate, pH 6.5.

### Example 8

### Bimolecular Tunable Affinity Ligand (TAL) Beacons for Protein Analysis

### Specific Ion Effects on Protein Binding

Specific ion effects were examined using the anchor sequence

### ⁵′NH₂-(CH₂)₆-CACGTAGCAG-Dabcyl³′

The hairpin-forming TAL construct (TAL1) was

### 5′ Cy3-GGTTG GTT TGG TTG G (HEG) CAACC TCT GCT ACG TG-3′

The underlined sequences represent arm sequences that form the stem structure of the hairpin in the absence of target. HEG is a hexaethylene glycol spacer.
TAL1 was designed to base pair with the anchor sequence under the appropriate ionic conditions. The molecule is quenched when in the hairpin form and unquenched (i.e., fluorescent) when in the quadruplex form. The effect of a 10 fold molar excess of α-thrombin on the solution fluorescence of a 277 nM solution of TAL1 was compared in KCl buffer and in LiCl buffer. The KCl buffer contained 12.5 mM Tris, pH 8.0, 10 mM KCl and 5 mM MgCl₂. The LiCl buffer contained 12.5 mM Tris, pH 8.0, 10 mM LiCl and 5 mM MgCl₂. The results demonstrated that the TAL in LiCl solution reached equilibrium much more rapidly than the TAL in KCl. See, FIG. 12, which provides a comparison of α-thrombin effect on bimolecular construct formed from TAL1 and Dabcyl anchor oligo in KCl buffer (12.5 mM Tris, pH 8.0, 10 mM KCl, 5 mM MgCl₂) and LiCl buffer (12.5 mM Tris, pH 8.0, 10 mM KCl, 5 mM MgCl₂).

### Example 9

### Comparing Tunable Affinity Ligand (TAL) Beacon Design

### Effect of Quenching Group

The Dabcyl anchor ⁵′NH₂-(CH₂)₆-CACGTAGCAG-Dabcyl³′ was compared to the Black Hole Quencher 2 anchor ⁵′NH₂-(CH₂)₆-CACGTAGCAG-BHQ2-³′ in terms of their effect on the fluorescence reporting behavior of TAL1. 277 nM of TAL1 was titrated with 1.5 fold molar excesses of the Dabcyl anchor and the BHQ2 anchor, and then with a 10:1 excess of α-thrombin. The results shown in FIG. 13 demonstrate the importance of choosing an anchor that shows sufficient but not excessive distance-dependent quenching. See, FIG. 13, providing a comparison of α-thrombin effect on TAL1 bimolecular construct formed with Dabcyl anchor and with BHQ2 anchor.

### Example 10

### Comparing Tunable Affinity Ligand (TAL) Beacon Design

### Effect of Flexible Linker

The TAL1 beacon, with an internal hexaethylene glycol linker was compared to the TAL2 beacon, which did not have an internal linker, as components of bimolecular constructs formed using the anchor ⁵′NH₂-(CH₂)₆-CACGTAGCAG-Dabcyl^{3′. 277} nM of TAL1 and TAL2 were titrated with a 1.5 fold molar excesses of the Dabcyl anchor, and then with a 10:1 excess of α-thrombin. The results shown in FIG. 14 illustrate that internal flexible linkers and other synthetic modifications can improve the performance of bimolecular constructs. See, FIG. 14, showing a comparison of α-thrombin effect on bimolecular construct formed with two different TAL probe constructs. The buffer was 12.5 mM Tris, pH 8.0, 10 mM KCl, 5 mM MgCl₂.

### Example 11

### Bimolecular Constructs Pre-Hybridized on Sepharose®-Coated Glass Slides

Experiments were performed using bimolecular constructs on CodeLink™ slides from GE Healthcare. These glass slides are coated with Sepharose®, and derivatized to allow covalent attachment of amino groups via Schiff's base chemistry. The probe DNA oligonucleotide in these experiments was:

5′ Cy3 -CACGCG AAC TAT ACA ACC TAC TAC CTC A
CGCGTG TC TGC TAC GTG - 3′

The 5′ amino anchor sequence was: 5′-6 amino-CAC GTA GCA G Dabcyl-3′, and the target DNA oligonucleotide was T GAG GTA GTA GGT TGT ATA GTT. In the first experiment, the probe oligomer and anchor oligomer were pre-hybridized at a ratio of 1:1. This pre-hybridized bimolecular construct was then spotted at a concentration of 10 μM using a GeneMachines Omnigrid microarraying robot and conjugated to the gel surface using the manufacturer's protocol. The slide was then washed extensively with SSC buffer (150 mM NaCl, 25 mM MgCl₂, 15 mM sodium citrate, pH 7). In FIG. 15 we compare the fluorescence intensity for spots obtained prior to incubation with target, and after 15 min incubation with 1 nM target oligo. See, FIG. 15, showing effects of pre-hybridizing probeacon oligo and anchor prior to spotting and conjugation. On the left hand side of the slide are spots monitored prior to the addition of target. On the right hand side are the same spots after incubation with 1 nM target oligo. The ratio of fluorescence before and after target addition was 3.1±0.1. See, FIG. 15, showing effects of pre-hybridizing probeacon oligo and anchor prior to spotting and conjugation.

### Example 12

### Bimolecular Constructs with Surface Attached Anchor Pre-Spotted on Sepharose®-Coated Glass Slides

Experiments were performed using bimolecular constructs on CodeLink slides from GE. The 5′ amino anchor oligo 5′-6 amino-CAC GTA GCA G Dabcyl-3′ was spotted and conjugated onto Code-link slides at a concentration of 10 μM. Using a 16-well gasket to allow multiple conditions on the same slide individual wells on the slide were incubated with variable concentration probeacon oligo for 15 minutes at 50° C. and then cooled to room temperature for 30 minutes. The slide was then rinsed with SSC buffer and the fluorescence monitored with the Gene-Pix scanner. The slide was then incubated with 1 nM target oligo for 15 min. Fluorescence data before and after target addition are shown for 100 fM pro-beacon spots in FIG. 16. See, FIG. 16, in which anchor oligo was spotted and conjugated onto Code-link slides at a concentration of 10 μM. The spots were washed with SSC buffer, incubated with 100 fM pro-beacon oligo, rinsed with SSC buffer (0.15 M NaCl, 0.015 M sodium citrate, pH 7) and the fluorescence was monitored with the scanner. The results are shown on the left hand side. On the right hand side are the same spots after incubation with 1 nM target oligo. The ratio of fluorescence before and after target addition was 30±5.

We show the ratio of fluorescence before and after target addition in FIG. 17, for pro-beacon concentrations ranging from 10 fM to 1 nM. See, FIG. 17, in which anchor oligo was spotted onto Code-link slides at a concentration of 10 μM, and NHS conjugation was performed using the manufacturer's protocol. The slide was washed extensively with SSC buffer, incubated with 10 fM to 1 nM pro-beacon oligo for 15 minutes at 50° C. and then cooled to room temperature for 30 minutes. The slide was then rinsed with SSC buffer and the fluorescence was monitored. The data represent average intensity ratios of for quadruplicate measurements of fluorescence before and after addition of 1 nM target oligo. Note that the fluorescence ratio decreases with increasing pro-beacon oligo concentration. These data demonstrate that our novel bimolecular construct can show enhancements in microarray experiments that are comparable to those observed in solution. Ordinary beacons must be spotted at relatively high concentrations in the micromolar range, thereby contributing to nonspecific binding/conjugation and unwanted background signal. In contrast, for bimolecular constructs the anchor can be spotted and conjugated at micromolar concentrations. Following this, the pro-beacon can be hybridized at femtomolar concentrations, thereby ameliorating nonspecific binding/conjugation and the resultant increase in fluorescence signal in the absence of target. As shown in FIG. 17, incubating pro-beacons at sub-picomolar concentrations gives much enhanced signal to background compared to incubating at higher (nanomolar) concentrations. See, FIG. 17.

### Example 13

### Bimolecular Constructs Attached by Both Strands of Duplex

Bimolecular constructs can also be attached by both strands. This embodiment is preferred since it allows extensive washing to remove nonspecifically associated, fluorescently labeled oligonucleotides. For example, the following strands may be attached in this manner:

probe oligo:
5′ Cy3 - CACGCG AAC TAT ACA ACC TAC TAC CTC A
CGCGTG TC TGC TAC GTG - C6 amino -3′

anchor-oligo: (5′ amino, DABCYL labeled): 5′-C6 amino-CAC GTA GCA G Dabcyl-3′ following coupling, and extensive washing, these strands are washed extensively to interact to bind

Target Oligo:
T GAG GTA GTA GGT TGT ATA GTT

In this embodiment, the probe oligo is pre-hybridized with the anchor oligonucleotide, and then attached via Schiff's base chemistry or other chemistry well-known to those skilled in the art to surfaces with closely spaced reactive groups. Periodate treated agarose is a preferred surface substrate because periodate treatment results in two closely spaced hydroxyl groups. Hydroxyl or epoxide coated glass slides also have a very high density of reactive groups and can be used to attach both strands simultaneously while maintaining hybridization.

### DESCRIPTION OF THE DRAWINGS

FIG. 1 is an illustration of a conventional Molecular Beacon that is a unimolecular construct with quencher and fluorophore on opposite ends of a hairpin-forming molecule. When the probe sequence in the loop of a molecular beacon binds to a target sequence a conformational reorganization occurs that restores the fluorescence of a quenched fluorophore. (See, for example, Marras, 2003)

FIG. 2 is a graph illustrating functional characterization of a molecular beacon by adding a complementary oligonucleotide target (See, for example, Marras, Kramer et al., 2003.)

FIG. 3 is an illustration of a bimolecular construct with 5′ fluorophore and 3′ linker for surface attachment and complementary anchor with 3′ quencher and 5′ linker for surface attachment.

FIG. 4 is an illustration of a Tunable Affinity Ligand (TAL) beacon with 5′ fluorophore and 3′ linker for surface attachment and complementary anchor with 3′quencher and 5′ linker.

A. The anchor sequence, with 3′ quencher is attached via a 5′ amino functionality to an amine-reactive surface.

B. The Tunable Affinity Ligand (TAL) functionality is hybridized to the anchor under conditions favoring hairpin formation (e.g. LiCl solution) and attached via a 3′ amino linker.

C. The Tunable Affinity Ligand (TAL) is switched to a protein-binding conformation (here, a quadruplex) under other conditions (e.g. KCl solution).

D. Protein binding to the active Tunable Affinity Ligand (TAL) conformation shifts the equilibrium toward that conformation.

FIG. 5 is a graph illustrating solution characterization at room temperature of a bimolecular construct with 5′ FAM labeled probe and complementary 3′ BHQ2 labeled anchor. (The fluorescence background of 150 μl of a 1 mM MgCl₂, 20 mM Tris-HCl, pH 8.0 solution was determined, using 491 nm as the excitation wavelength and 515 as the emission wavelength. 10 μl of 1 μM FAM labeled DNA hairpin (HP2) was added to this solution and the new level of fluorescence was recorded. A two-fold molar excess of quencher labeled anchor DNA oligonucleotide was added and the decrease in fluorescence was monitored until it reached a stable level. Finally, a five-fold molar excess of target DNA oligonucleotide was added and the increase in fluorescence was monitored.)

FIG. 6 is a graph illustrating solution characterization at room temperature of a bimolecular construct comprising a 5′ FAM labeled 2′O-methyl anchor RNA and a 3′ Dabcyl labeled 2′ O-methyl RNA probe complementary in the hairpin loop region to let 7B RNA. (The background of a solution of 4 mM MgCl₂, 20 mM Tris-HCl, pH 8.0 solution was determined, using 491 nm as the excitation wavelength and 515 as the emission wavelength. Anchor was added to a concentration of 800 nM, followed by the addition of let 7B probe to a concentration of 2 μM. Finally, let 7B target RNA was added to a concentration of 8 μM.)

FIG. 7 is a graph illustrating solution characterization of the temperature dependence of a bimolecular construct comprising 800 nM 5′ FAM labeled 2′O-methyl anchor RNA and 2 μM 3′ dabcyl labeled 2′ O-methyl let 7B RNA probe in the presence of let 7A (two mismatches), let 7B (fully complementary), let 7C (single mismatch) and let 7F (three mismatches) target molecules at concentrations of 8 μM each. (The solution included 4 mM MgCl₂, 20 mM Tris-HCl, pH 8.0. Fluorescence was monitored with 491 nm as the excitation wavelength and 515 nm as the emission wavelength.)

FIG. 8 is a graph illustrating the effect of a 1.5 fold excess of complement on the fluorescence intensity of the TAL2 beacon. (The TAL2 concentration was 277 nM. The solid circles refer to results in 100 mM LiCl, 10 mM Tris, pH 8.0. The hollow circles are for results in 100 mM KCl, 10 mM Tris, pH 8.0.)

FIG. 9 is a graph illustrating the results of adding a 10 fold excess of α-thrombin on the fluorescence intensity of the TAL2 beacon construct. (The total concentration of TAL2 was 277 nM. The solution contained 10 mM KCl, 5 mM MgCl₂, and 12.5 mM Tris Acetate, pH 6.5.)

FIG. 10 is a graph illustrating α-thrombin concentration dependence of the fluorescence from the TAL2 beacon. (The TAL concentration was 277 nM. The legends beside the graph show the ratio of added α-thrombin to TAL. The unquenched fluorescence refers to the intensity of TAL2 in the absence of added anchor or target. The solution contained 10 mM KCl, 5 mM MgCl₂, and 12.5 mM Tris Acetate, pH 6.5.)

FIG. 11 is a graph illustrating a comparison of the effects of 100:1 molar ratios of α-, β- and γ-thrombin on the dilution-corrected fluorescence of the TAL2 beacon. (The total concentration of aptamer was 277 nM. The solution contained 10 mM KCl, 5 mM MgCl₂, and 12.5 mM Tris Acetate, pH 6.5.)

FIG. 12 is a graph illustrating a comparison of α-thrombin effect on bimolecular construct formed from TAL1, and Dabcyl Anchor Oligo in KCl buffer (12.5 mM Tris, pH 8.0, 10 mM KCl, 5 mM MgCl₂) and LiCl buffer (12.5 mM Tris, pH 8.0, 10 mM KCl, 5 mM MgCl₂).

FIG. 13 is a graph illustrating a comparison of α-thrombin effect on TAL1 bimolecular construct with Dabcyl anchor and with BHQ2 anchor. (Buffer was 12.5 mM Tris, pH 8.0, 10 mM KCl, 5 mM MgCl₂)).

FIG. 14 is a graph illustrating a comparison of α-thrombin effect on bimolecular construct with two different aptamer constructs. (TAL1 contained a flexible hexaethylene glycol spacer. TAL2 had no spacer. Buffer was 12.5 mM Tris, pH 8.0, 10 mM KCl, 5 mM MgCl₂)).

FIG. 15 is an illustration of effects of pre-hybridizing pro-beacon oligo and anchor prior to spotting and conjugation. (On the left hand side are spots monitored prior to the addition of target. On the right hand side are the same spots after incubation with 1 nM target oligo. The ratio of fluorescence before and after target addition was 3.1±0.1.)

FIG. 16 is an illustration of anchor oligo that was spotted and conjugated onto Code-link slides at a concentration of 10 μM. (The spots were washed with SSC buffer, incubated with 100 fM pro-beacon oligo, rinsed with SSC buffer (0.15 M NaCl, 0.015 M sodium citrate, pH 7) and the fluorescence was monitored with the scanner. The results are shown on the left hand side. On the right hand side are the same spots after incubation with 1 nM target oligo. The ratio of fluorescence before and after target addition was 30±5.)

FIG. 17 is a graph of anchor oligo that was spotted onto Code-link slides at a concentration of 10 μM, and Schiff's base conjugation was performed using the manufacturer's protocol. (The slide was washed extensively with SSC buffer, incubated with 10 fM to 1 nM pro-beacon oligo for 15 minutes at 50° C. and then cooled to room temperature for 30 minutes. The slide was then rinsed with SSC buffer and the fluorescence was monitored. The data represent average intensity ratios of for quadruplicate measurements of fluorescence before and after addition of 1 nM target oligo.)

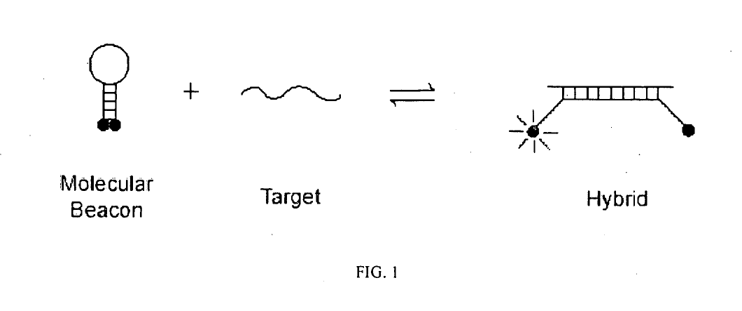

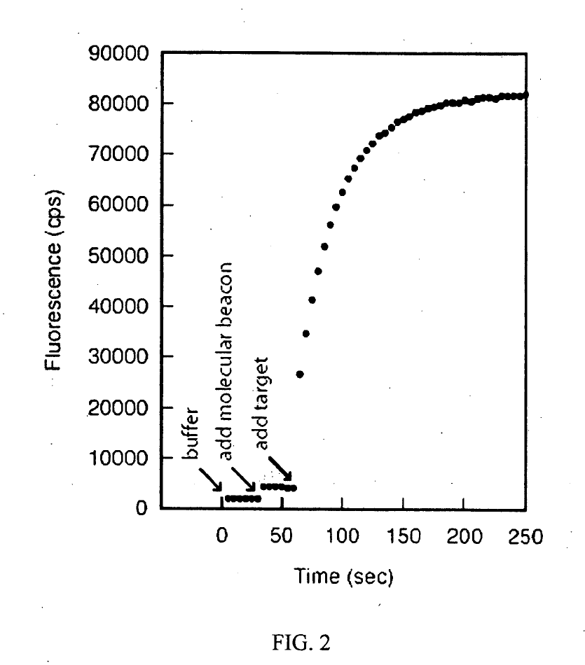

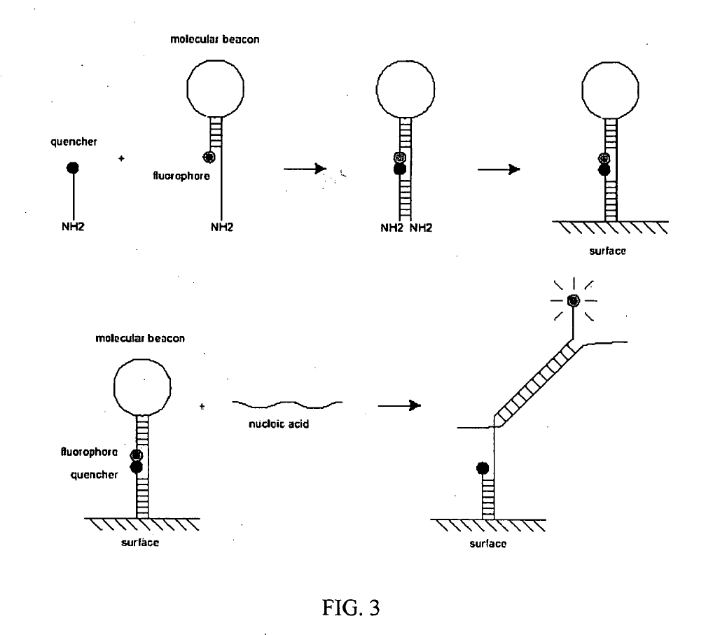

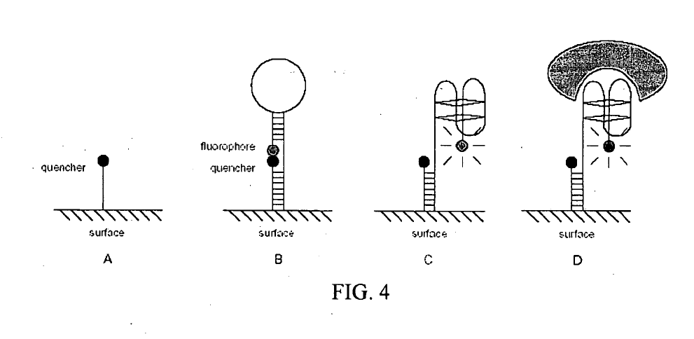

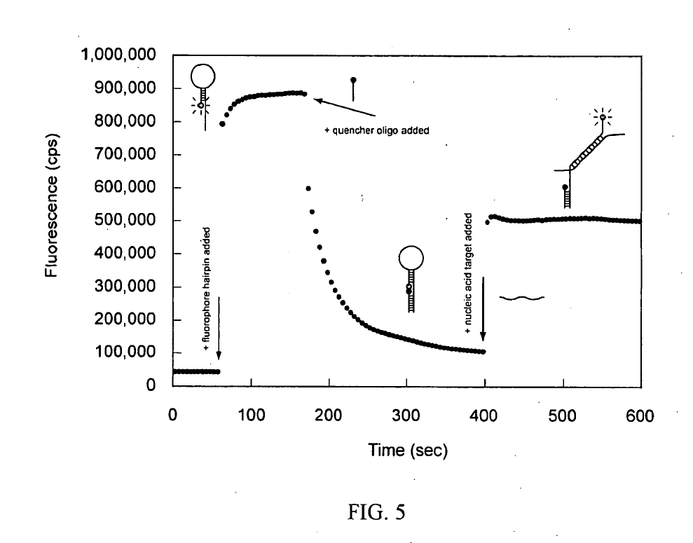

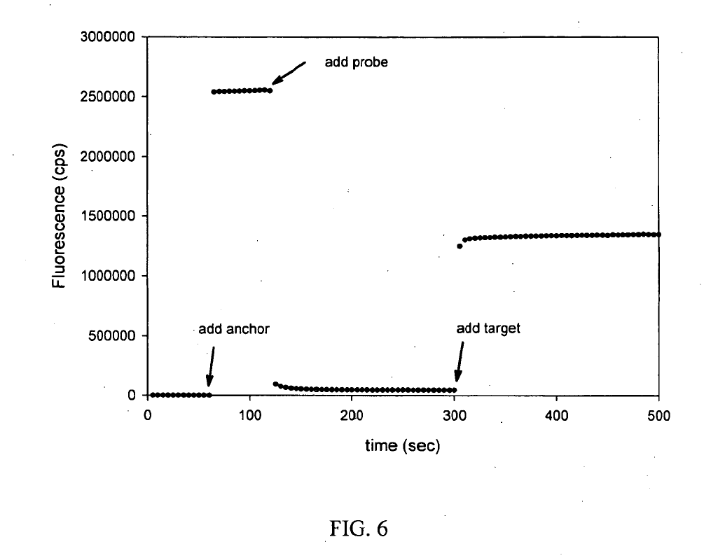

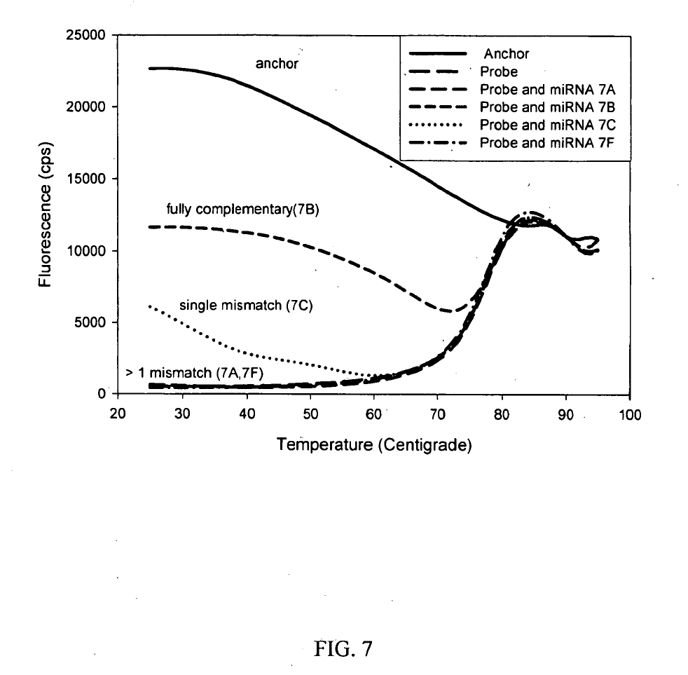

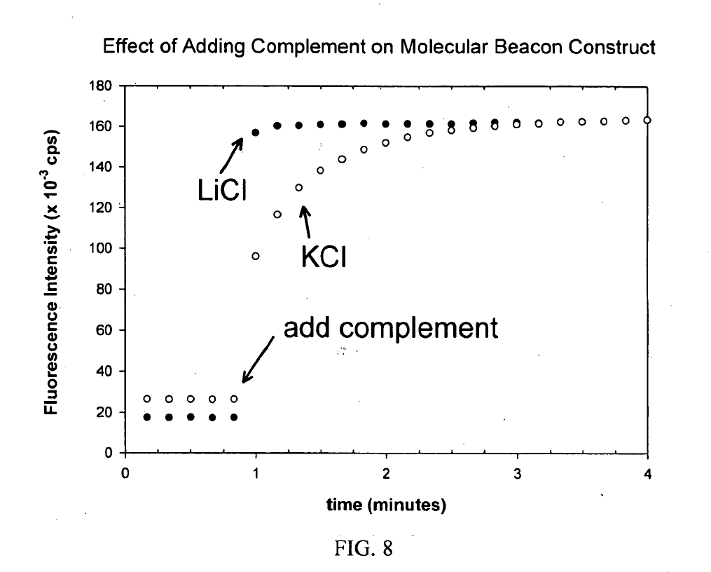

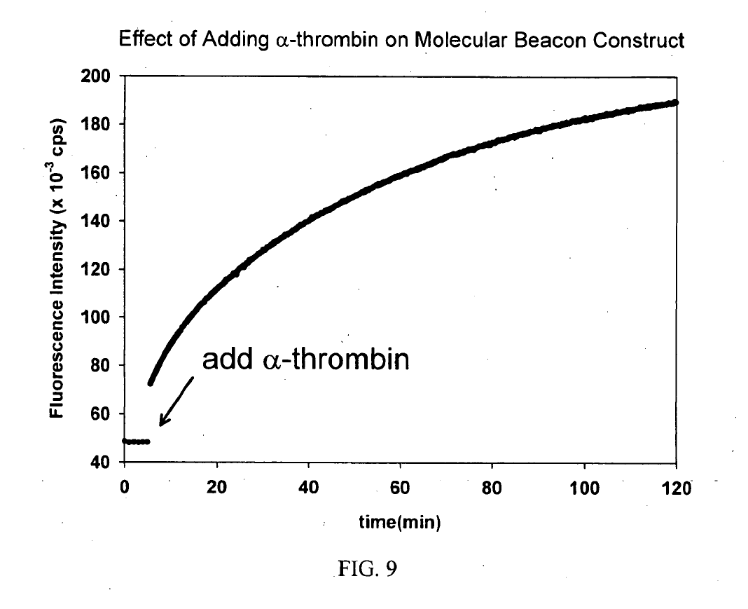

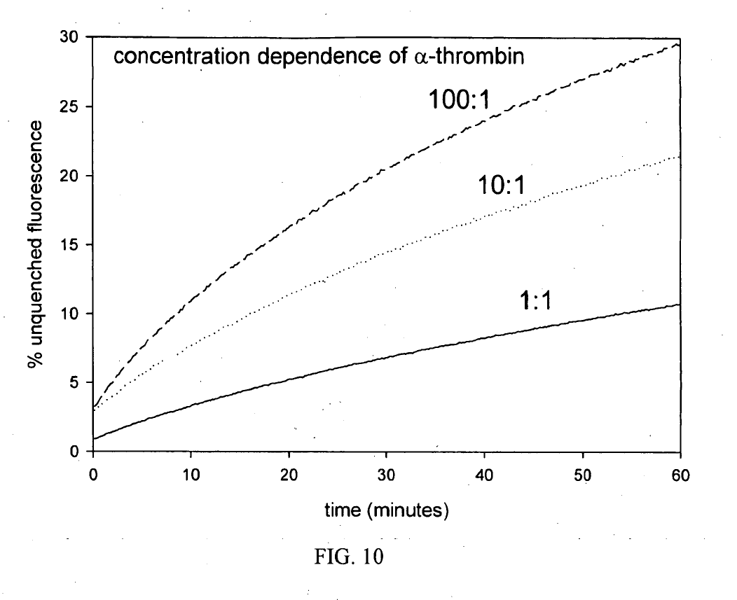

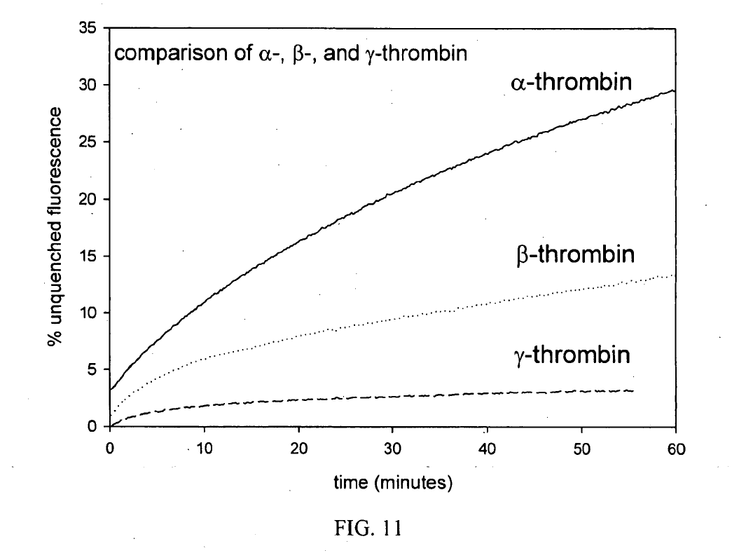

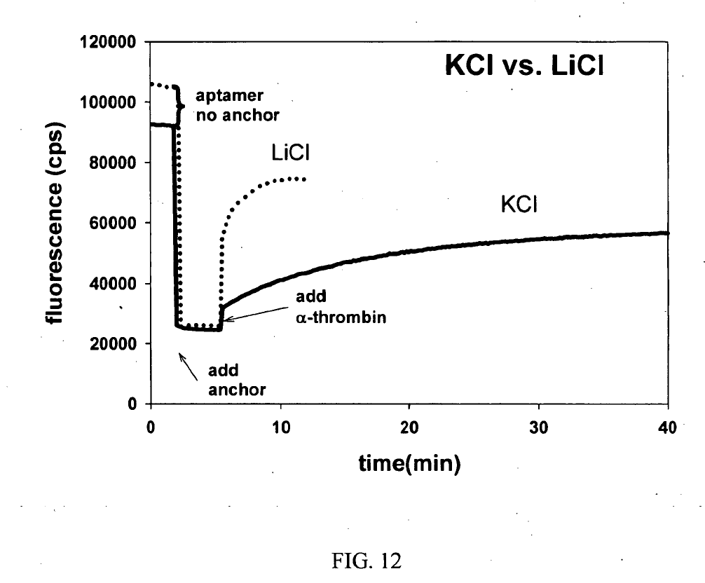

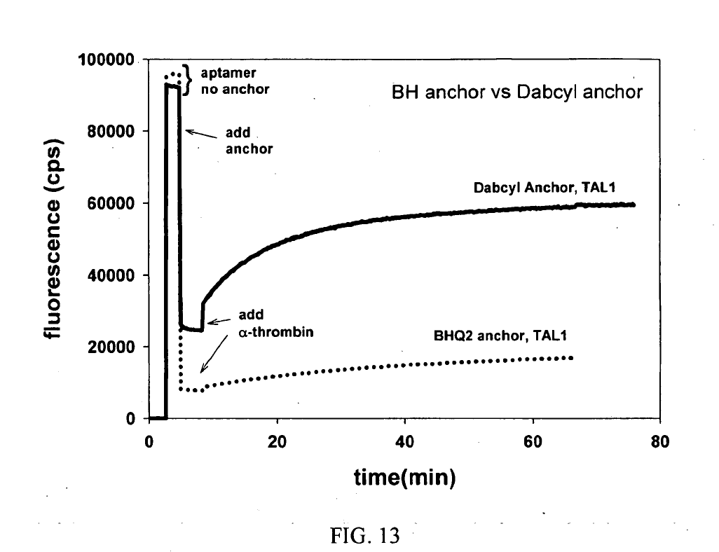

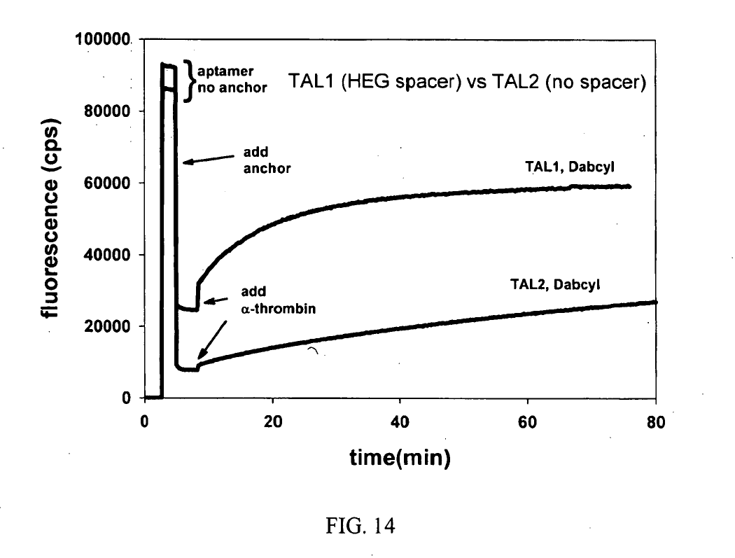

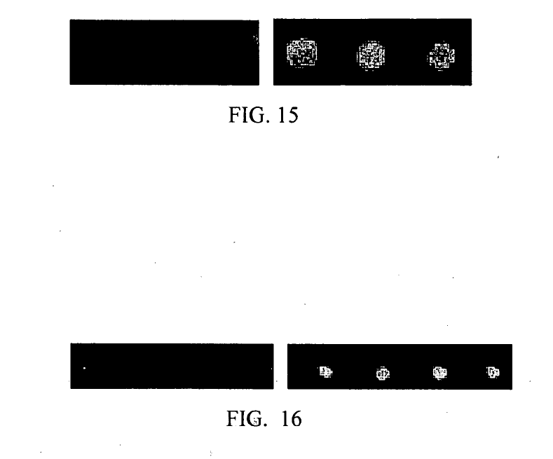

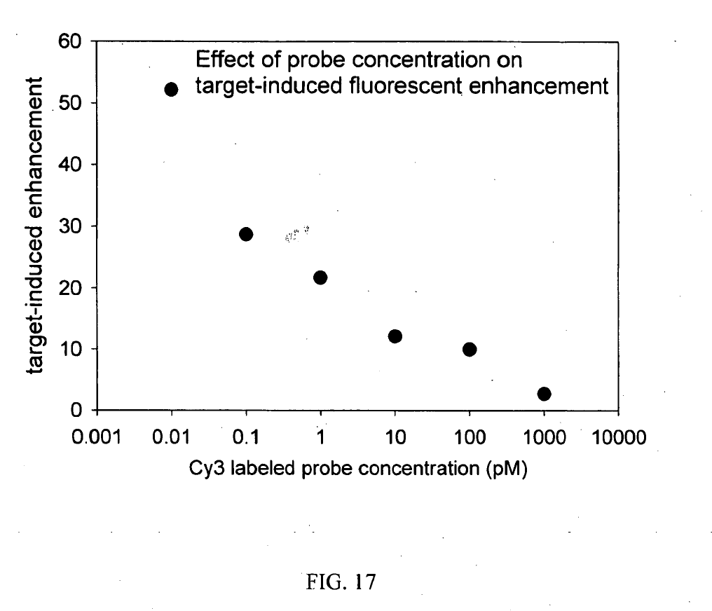

### LIST OF REFERENCED ARTICLES

Afanassiev, V., Hanemann, V., and Wolfl, S. (2000). Preparation of DNA and protein micro arrays on glass slides coated with an agarose film. Nucleic Acids Res 28, E66.
Beaucage, S. L. (2001). Strategies in the preparation of DNA oligonucleotide arrays for diagnostic applications. Curr Med Chem 8, 1213-1244.
Bonnet, G., Tyagi, S., Libchaber, A., and Kramer, F. R. (1999). Thermodynamic basis of the enhanced specificity of structured DNA probes. Proc Natl Acad Sci USA 96, 6171-6176.
Bratu, D. P., Cha, B. J., Mhlanga, M. M., Kramer, F. R., and Tyagi, S. (2003). Visualizing the distribution and transport of mRNAs in living cells. Proc Natl Acad Sci USA 100, 13308-13313.
Brody, E. N., and Gold, L. (2000). Aptamers as therapeutic and diagnostic agents. J Biotechnol 74, 5-13.
Cogoi, S., Quadrifoglio, F., and Xodo, L. E. (2004). G-rich oligonucleotide inhibits the binding of a nuclear protein to the Ki-ras promoter and strongly reduces cell growth in human carcinoma pancreatic cells. Biochemistry 43, 2512-2523.
Culha, M., Stokes, D. L., Griffin, G. D., and Vo-Dinh, T. (2004). Application of a miniature biochip using the molecular beacon probe in breast cancer gene BRCA1 detection. Biosens Bioelectron 19, 1007-1012.
Dapic, V., Abdomerovic, V., Marrington, R., Peberdy, J., Rodger, A., Trent, J. O., and Bates, P. J. (2003). Biophysical and biological properties of quadruplex oligodeoxyribonucleotides. Nucleic Acids Res 31, 2097-2107.
Decher, G. (1997). Fuzzy nanoassemblies: toward layered polymeric multicomposites. Science 277, 1232-1237.
Du, H., Disney, M. D., Miller, B. L., and Krauss, T. D. (2003). Hybridization-based unquenching of DNA hairpins on au surfaces: prototypical “molecular beacon” biosensors. J Am Chem Soc 125, 4012-4013.
Famulok, M., and Mayer, G. (1999). Aptamers as tools in molecular biology and immunology. Curr Top Microbiol Immunol 243, 123-136.
Hamaguchi, N., Ellington, A., and Stanton, M. (2001). Aptamer beacons for the direct detection of proteins. Anal Biochem 294, 126-131.
Jing, N., Li, Y., Xu, X., Sha, W., Li, P., Feng, L., and Tweardy, D. J. (2003). Targeting Stat3 with G-quartet oligodeoxynucleotides in human cancer cells. DNA Cell Biol 22, 685-696.
Kartalov, E. P., Unger, M. A., and Quake, S. R. (2003). Polyelectrolyte surface interface for single-molecule fluorescence studies of DNA polymerase. Biotechniques 34, 505-510.
Khrapko, K. R., Lysov Yu, P., Khorlin, A. A., Ivanov, I. B., Yershov, G. M., Vasilenko, S. K., Florentiev, V. L., and Mirzabekov, A. D. (1991). A method for DNA sequencing by hybridization with oligonucleotide matrix. DNA Seq 1, 375-388.
Khrapko, K. R., Lysov Yu, P., Khorlyn, A. A., Shick, V. V., Florentiev, V. L., and Mirzabekov, A. D. (1989). An oligonucleotide hybridization approach to DNA sequencing. FEBS Lett 256, 118-122.
Lin, Y. C., Shih, J. W., Hsu, C. L., and Lin, J. J. (2001). Binding and partial denaturing of G-quartet DNA by Cdcl3p of Saccharomyces cerevisiae. J Biol Chem 276, 47671-47674.
Lofas, S., and Johnsson, B. (1990). A Novel hydrogel matrix on gold surfaces in surface plasmon resonance sensors for fast and efficient covalent immobilization of ligands. Journal of the Chemical Society, Chemical Communications 21, 1526-1528.
Marras, S. A. (2003a) Development of molecular beacons for nucleic acid detection, Ph.D., University of Leiden, Leiden.
Marras, S. A. (2005). Fluorescent Energy Transfer: Nucleic Acid Probes and Protocols (Totowa, Humana Press).
Marras, S. A., Kramer, F. R., and Tyagi, S. (1999). Multiplex detection of single-nucleotide variations using molecular beacons. Genet Anal 14, 151-156.
Marras, S. A., Kramer, F. R., and Tyagi, S. (2002). Efficiencies of fluorescence resonance energy transfer and contact-mediated quenching in oligonucleotide probes. Nucleic Acids Res 30, e122.
Marras, S. A., Kramer, F. R., and Tyagi, S. (2003a). Genotyping SNPs with molecular beacons. Methods Mol Biol 212, 111-128.
Marras, S. A. E. (2003b). Development of molecular beacons for nucleic acid detection.
Marras, S. A. E., Kramer, F. R., and Tyagi, S. (2003b). Genotyping single-nucleotide polymorphisms with molecular beacons. Methods Mol Biol 212, 111-128.
Rangan, A., Fedoroff, O. Y., and Hurley; L. H. (2001). Induction of duplex to G-quadruplex transition in the c-myc promoter region by a small molecule. J Biol Chem 276, 4640-4646.
Siddiqui-Jain, A., Grand, C. L., Bearss, D. J., and Hurley, L. H. (2002). Direct evidence for a G-quadruplex in a promoter region and its targeting with a small molecule to repress c-MYC transcription. Proc Natl Acad Sci USA 99, 11593-11598.
Steel, A. B., Levicky, R. L., Herne, T. M., and Tarlov, M. J. (2000). Immobilization of nucleic acids at solid surfaces: effect of oligonucleotide length on layer assembly. Biophys J 79, 975-981.
Steemers, F. J., Ferguson, J. A., and Walt, D. R. (2000). Screening unlabeled DNA targets with randomly ordered fiber-optic gene arrays. Nat Biotechnol 18, 91-94.
Szuhai, K., Ouweland, J., Dirks, R., Lemaitre, M., Truffert, J., Janssen, G., Tanke, H., Holme, E., Maassen, J., and Raap, A. (2001). Simultaneous A8344G heteroplasmy and mitochondrial DNA copy number quantification in myoclonus epilepsy and ragged-red fibers (MERRF) syndrome by a multiplex molecular beacon based real-time fluorescence PCR. Nucleic Acids Res 29, E13.
Timofeev, E., Kochetkova, S. V., Mirzabekov, A. D., and Florentiev, V. L. (1996). Regioselective immobilization of short oligonucleotides to acrylic copolymer gels. Nucleic Acids Res 24, 3142-3148.
Tyagi, S., Bratu, D. P., and Kramer, F. R. (1998). Multicolor molecular beacons for allele discrimination. Nat Biotechnol 16, 49-53.
Tyagi, S., and Kramer, F. R. (1996). Molecular beacons: probes that fluoresce upon hybridization. Nat Biotechnol 14, 303-308.
Wang, H., Li, J., Liu, H., Liu, Q., Mei, Q., Wang, Y., Zhu, J., He, N., and Lu, Z. (2002). Label-free hybridization detection of a single nucleotide mismatch by immobilization of molecular beacons on an agarose film. Nucleic Acids Res 30, e61.
Wilson, D. S., and Szostak, J. W. (1999). In vitro selection of functional nucleic acids. Annu Rev Biochem 68, 611-647.
Yao, G., and Tan, W. (2004). Molecular-beacon-based array for sensitive DNA analysis. Anal Biochem 331, 216-223.

What is claimed is:

1. An immobilized bimolecular construct comprising a support, a first oligonucleotide having a first end and a second end and a second oligonucleotide having a first end and a second end wherein:
a) the first oligonucleotide is labeled at the first end with one of a fluorophore or a quencher and immobilized at the second end to the support;
b) the second oligonucleotide is labeled at the first end with the other of a fluorophore or a quencher and hybridized at the second end to the first oligonucleotide;
c) at least one of the first oligonucleotide, the second oligonucleotide or a combination of the first and second oligonucleotides is capable of specifically binding to a target molecule.

2. The immobilized bimolecular construct of claim 1 wherein the first oligonucleotide or the second oligonucleotide is a hairpin-forming oligonucleotide comprising a defined sequence segment capable of specifically binding to the target molecule.

3. The immobilized bimolecular construct of claim 1 or 2 wherein the target molecule is selected from the group consisting of lipids, proteins and nucleic acids,

4. (canceled)

5. (canceled)

6. (canceled)

7. (canceled)

8. (canceled)

9. The immobilized bimolecular construct of claim 1 wherein the at least one hairpin-forming oligonucleotide comprises a probe sequence capable of specifically hybridizing to a nucleic acid target.

10. The immobilized bimolecular construct of claim 1 wherein the at least one hairpin-forming oligonucleotide comprises a target-binding region capable of specifically binding to a nonoligonucleotide molecule.

11. An immobilized bimolecular construct comprising a solid support, a first oligonucleotide having a first end and a second end and a second oligonucleotide having a first end and a second end wherein said construct comprises defined sequence segments capable of hybridizing to form a hairpin structure and wherein:
a) the first oligonucleotide is labeled at the first end with one of a fluorophore or quencher and attached at the second end to a solid support;
b) the second oligonucleotide is labeled at the first end with the other of a fluorophore or quencher and hybridized at the second end to the first oligonucleotide; and
c) the hairpin structure is capable of specifically binding to a target molecule.

12. The immobilized bimolecular construct of claim 11 wherein the first oligonucleotide or the second oligonucleotide is capable of forming a hairpin structure.

13. The immobilized bimolecular construct of claim 11 or 12 wherein the hairpin structure is capable of specifically binding to a lipid, protein or nucleic acid target.

14. (canceled)

15. (canceled)

16. (canceled)

17. (canceled)

18. (canceled)

19. An immobilized bimolecular construct as recited in claim 1 through 18 wherein either said first oligonucleotide or said second oligonucleotide is attached to the support.

20. An immobilized bimolecular construct as recited in claim 1 through 19 wherein both said first oligonucleotide and said second oligonucleotide are attached to the support.

21. The immobilized bimolecular construct of claim 19 wherein said first oligonucleotide or said second oligonucleotide is attached to the support by covalent means.

22. The immobilized bimolecular construct of claim 20 wherein at least one of said first oligonucleotide or said second oligonucleotide is attached to the support by covalent means.

23. The immobilized bimolecular construct of claim 20 wherein both said first oligonucleotide and said second oligonucleotide are attached to the support by covalent means.

24. An immobilized bimolecular construct as recited in claim 1 through 23 wherein the target molecule is selected from the group consisting of natural or synthetic peptide, protein or nucleic acid molecules, natural or synthetic carbohydrates or small molecule sugars, natural or synthetic small molecules or ions, biomolecular complexes, cell surfaces, viruses or other complex biological targets, and naturally occurring or synthetic mimetics, conjugates, derivatives or analogs thereof.

25. An immobilized bimolecular construct as recited in claim 24 wherein the natural or synthetic small molecule comprises a drug, a pharmacophore, a metabolite, a metal ion or a toxin.

26. An immobilized bimolecular construct as recited in claim 24 wherein the biomolecular complex comprises a ribonucleoprotein complex, a protein complex or a protein-carbohydrate complex.

27. An immobilized bimolecular construct as recited in claim 1 through 26 wherein the first oligonucleotide or the second oligonucleotide comprises an aptamer sequence that specifically recognizes a protein target.

28. An immobilized bimolecular construct comprising a solid support, a first oligonucleotide having a first end and a second end and a second oligonucleotide having a first end and a second end wherein:
a) the first oligonucleotide is labeled at the first end with one of a fluorophore or quencher;
b) the second oligonucleotide is labeled at the first end with the other of a fluorophore or quencher;
c) at least the first oligonucleotide or the second oligonucleotide is attached at its second end to a solid support;
d) the first oligonucleotide or the second oligonucleotide is capable of forming a hairpin-loop structure; and
e) the first oligonucleotide and the second oligonucleotide are hybridizably linked in a manner that positions the labeled end of the first oligonucleotide within energy transfer distance of the labeled end of the second oligonucleotide.

29. (canceled)

30. (canceled)

31. (canceled)

32. (canceled)

33. (canceled)

34. (canceled)

35. (canceled)

36. (canceled)

37. An immobilized bimolecular construct as recited in claim 1 through 26 wherein the first oligonucleotide or the second oligonucleotide comprises an aptamer sequence that specifically recognizes a carbohydrate or small molecule sugar.

38. (canceled)

39. (canceled)

40. (canceled)

41. (canceled)

42. (canceled)

43. (canceled)

44. (canceled)

45. (canceled)

46. An immobilized bimolecular construct as recited in claim 1 through 26 wherein the first oligonucleotide or the second oligonucleotide comprises an aptamer sequence that specifically recognizes a natural or synthetic small molecule or ion.

47. An immobilized bimolecular construct as recited in claim 46 wherein said natural or synthetic small molecule or ion comprises a drug, a pharmacophore, a metabolite, a metal ion or a toxin.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. An immobilized bimolecular construct as recited in claim 1 through 26 wherein the first oligonucleotide or the second oligonucleotide comprises an aptamer sequence that specifically recognizes biomolecular complexes (e.g. ribonucleoprotein complexes, protein complexes and protein-carbohydrate complexes), cell surfaces, viruses, and other complex biological targets.

60. An immobilized bimolecular construct as recited in claim 59 wherein the biomolecular complex comprises a ribonucleoprotein complex, a protein complex or a protein-carbohydrate complex.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)
